(19) United States
(12) Patent Application Publication
(10) Pub. No.: US 2026/0007710 A1
(43) Pub. Date: Jan. 8, 2026

(54) PRODUCTION PROCESS FOR CONTROLLING THE ACTIVE INGREDIENTS OF Cistanche deserticola Ma

(71) Applicant: Sichuan JUYUAN Chinese MEDICINE Herbal Pieces Co., Ltd., Dazhou (CN)

(72) Inventors: Zhongbiao Liao, Dazhou (CN); Hongjun Tang, Dazhou (CN); Haozhi Zhao, Dazhou (CN); Guanghua Lv, Dazhou (CN); Guangren Zhang, Dazhou (CN); Jiancheng Lin, Dazhou (CN); Li Xiao, Dazhou (CN)

(21) Appl. No.: 18/762,603

(22) Filed: Jul. 2, 2024

(51) Int. Cl. A61K 36/64

(52) U.S. Cl. CPC A61K 36/64; A61K 2236/15; A61K 2236/51; A61K 2236/53

(57) ABSTRACT

A production process for controlling the active ingredients of Cistanche deserticola Ma is provided, including: (1) crushing and screening the raw material of Cistanche deserticola Ma, mixing the powder of Cistanche deserticola Ma with a solvent, and then performing ultrasonic extraction; (2) performing centrifugation on the extraction solution, then performing membrane separation on the supernatant; (3) concentrating the solution containing the active ingredients of Cistanche deserticola Ma under vacuum, then mixing the concentrated solution with excipients and stirring evenly to prepare a controlled release formulation; (4) drying the prepared controlled release formulation of the active ingredients of Cistanche deserticola Ma to obtain a dried controlled release product of the active ingredients of Cistanche deserticola Ma. By adopting ultrasonic extraction, centrifugal impurity removal, membrane separation, vacuum concentration, and drying, the efficient extraction and purification of active ingredients in Cistanche deserticola Ma have been achieved, improving product quality and stability.

### TECHNICAL FIELD

The present disclosure relates to the technical field of traditional Chinese medicine extraction, in particular to a production process for controlling the active ingredients of Cistanche deserticola Ma.

### BACKGROUND

Cistanche deserticola Ma, also known as cunyun, desert cistanche, or chagan gaoya (Mongolian), is a parasitic plant that parasitizes the roots of the tree Haloxylon ammodendron in desert, absorbing nutrients and water from its host. The characteristics of Cistanche deserticola Ma are: fleshy stem, long cylindrical or slightly flattened at the bottom, slightly curved, 3-375px long, thicker at the bottom, with a diameter of 5-375px, and gradually tapering upwards, with a diameter of 2-375px. Cistanche deserticola Ma is known as “ginseng in desert” and has extremely high medicinal value, which is a precious traditional Chinese medicinal materials, with the functions of tonifying kidney Yang, nourishing essence and blood, and moistening the intestines. It is mainly used for treatment of deficiency and decline of kidney Yang, impotence caused by insufficient essence and blood, nocturnal emission, white turbidity, frequent and dripping urine, low back pain and weak feet, tinnitus and blurred vision, menstrual delay, uterine cold infertility, intestinal dryness and constipation, etc. However, the extraction of effective components from Cistanche deserticola Ma is difficult, and traditional extraction methods often lead to degradation and loss of effective components. In addition, traditional production processes are difficult to achieve precise control of effective ingredient release, which affects the stability and persistence of drug efficacy. Therefore, studying an efficient and stable method for extracting effective components from Cistanche deserticola Ma has important practical significance and application value.

### SUMMARY

In view of this, the present disclosure provides a production process for controlling the active ingredients of Cistanche deserticola Ma, aiming to solve the problem of degradation and loss of active ingredients that often occur in current technologies.

A production process for controlling the active ingredients of Cistanche deserticola Ma, including the following steps:
(1) crushing and screening the raw material of Cistanche deserticola Ma to obtain powder of Cistanche deserticola Ma; mixing the powder of Cistanche deserticola Ma with a solvent, and then performing ultrasonic extraction to obtain extraction solution;
(2) performing centrifugation on the extraction solution to remove impurities and insoluble substances to obtain supernatant, then performing membrane separation on the supernatant to obtain a solution containing active ingredients of Cistanche deserticola Ma;
(3) performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) drying the prepared controlled release formulation of the active ingredients of Cistanche deserticola Ma to obtain a dried controlled release product of the active ingredients of Cistanche deserticola Ma.

Optionally, the extraction temperature of the ultrasonic extraction is 40-60° C., and the extraction time is 30-60 minutes.

Optionally, the membrane material selected for the membrane separation is one of polysulfone, polyethersulfone, or polyamide.

Optionally, the pore size of the membrane separation is selected between 0.1 and 10 microns.

Optionally, the centrifugal speed is 4000 rpm/min.

Optionally, the concentration temperature of the vacuum concentration is 65° C., and the vacuum degree during concentration is above −0.08 Mpa.

Optionally, the drying temperature is 70° C., and the vacuum degree during drying is above −0.08 Mp.

Optionally, the solvent is one selected from the group consisting of water, ethanol, methanol, ethyl acetate, n-butanol, chloroform, and dimethyl sulfoxide.

Compared with existing technology, the advantageous effect of the present disclosure lies in that: the present disclosure provides a production process for controlling the active ingredients of Cistanche deserticola Ma, which achieves efficient extraction and purification of the active ingredients of Cistanche deserticola Ma through steps such as ultrasonic extraction, centrifugal impurity removal, membrane separation, vacuum concentration, and drying, improving product quality and stability. By using ultrasonic extraction technology, it can improve the extraction efficiency and amount of active ingredients in Cistanche deserticola Ma under lower temperature and time conditions, reducing energy consumption and loss of active ingredients. By centrifugation and membrane separation steps, impurities and insoluble substances in the extraction solution can be effectively removed, improving the purity and quality stability of the product. By using vacuum concentration technology, a solution containing the active ingredients of Cistanche deserticola Ma can be quickly concentrated at lower temperatures, reducing the thermal decomposition and oxidation losses of the active ingredients, and improving the stability and shelf life of the product. By controlling the preparation of release formulations, slow release and long-lasting effects of the active ingredients in Cistanche deserticola Ma can be achieved, improving the efficacy and utilization of the product. By adopting drying technique, the controlled release product of active ingredients in Cistanche deserticola Ma can be quickly dried under lower temperature and vacuum conditions, ensuring the quality and stability of the product.

### BRIEF DESCRIPTION OF THE DRAWINGS

The following will provide a clear and complete description of the technical solutions in the embodiments of the present disclosure. It is obvious that the described embodiments are only a part of the embodiments of the present disclosure, not all of them. Based on the embodiments in the present disclosure, all other embodiments obtained by ordinary skilled person in the art without creative labor fall within the scope of the present disclosure.

In some embodiments of this application, a production process for controlling the active ingredients of Cistanche deserticola Ma is provided by the present embodiment, which includes the following steps:
(1) Crushing and screening the raw material of Cistanche deserticola Ma to obtain powder of Cistanche deserticola Ma; mixing the powder of Cistanche deserticola Ma with a solvent, and then performing ultrasonic extraction to obtain extraction solution;
(2) Performing centrifugation on the extraction solution to remove impurities and insoluble substances to obtain supernatant, then performing membrane separation on the supernatant to obtain a solution containing active ingredients of Cistanche deserticola Ma;
(3) Performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) Drying the prepared controlled release formulation of the active ingredients of Cistanche deserticola Ma to obtain a dried controlled release product of the active ingredients of Cistanche deserticola Ma.

In the present embodiment, the extraction temperature of the ultrasonic extraction is 40-60° C., and the extraction time is 30-60 minutes.

It can be understood that using ultrasonic extraction technology can improve the extraction efficiency and amount of active ingredients in Cistanche deserticola Ma under lower temperature and time conditions, reducing energy consumption and loss of active ingredients.

In the present embodiment, the membrane material selected for the membrane separation is one of polysulfone, polyethersulfone, or polyamide.

In the present embodiment, the pore size of the membrane separation is selected between 0.1 and 10 microns.

In the present embodiment, the centrifugal speed is 4000 rpm/min.

It can be understood that through the steps of centrifugation and membrane separation, impurities and insoluble substances in the extraction solution can be effectively removed, improving the purity and quality stability of the product.

In the present embodiment, the concentration temperature of the vacuum concentration is 65° C., and the vacuum degree during concentration is above −0.08 Mpa.

It can be understood that using vacuum concentration technology can quickly concentrate solutions containing the active ingredients of Cistanche deserticola Ma under lower temperature, reducing the thermal decomposition and oxidation losses of the active ingredients, and improving the stability and shelf life of the product.

In the present embodiment, the drying temperature is 70° C., and the vacuum degree during drying is above −0.08 Mp.

It can be understood that the use of drying technology can quickly control the release of effective ingredients in Cistanche deserticola Ma under lower temperature and vacuum conditions, ensuring the quality and stability of the product.

In the present embodiment, the solvent is one selected from the group consisting of water, ethanol, methanol, ethyl acetate, n-butanol, chloroform, and dimethyl sulfoxide.

The following will provide a further detailed explanation of the above anti-dizziness product through embodiments, but the embodiments are not a limitation of the present disclosure.

The fresh Cistanche deserticola Ma from the same batch are dried and divided evenly into 8 parts, and labeled as groups 1-8, wherein the groups 1-6 are embodiment 1-embodiment 6, and the groups 7-8 are comparative examples 1-2.

### EMBODIMENT 1

(1) Crushing and screening the raw material of Cistanche deserticola Ma to obtain powder of Cistanche deserticola Ma, mixing the powder of Cistanche deserticola Ma with appropriate amount of water, and then performing ultrasonic extraction to obtain extraction solution, wherein the extraction temperature of the ultrasonic extraction is 40° C., and the extraction time is 60 minutes.
(2) Performing centrifugation on the extraction solution to remove impurities and insoluble substances to obtain supernatant, then performing membrane separation on the supernatant to obtain a solution containing active ingredients of Cistanche deserticola Ma, wherein the centrifugal speed is 4000 rpm/min; the membrane material selected for the membrane separation is one of polysulfone; and the pore size of the membrane separation is 0.8 microns.
(3) Performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) Drying the prepared controlled release formulation of the active ingredients of Cistanche deserticola Ma to obtain a dried controlled release product of the active ingredients of Cistanche deserticola Ma.

### EMBODIMENT 2

(1) Crushing and screening the raw material of Cistanche deserticola Ma to obtain powder of Cistanche deserticola Ma, mixing the powder of Cistanche deserticola Ma with appropriate amount of water, and then performing ultrasonic extraction to obtain extraction solution, wherein the extraction temperature of the ultrasonic extraction is 50° C., and the extraction time is 30 minutes.
(2) Performing centrifugation on the extraction solution to remove impurities and insoluble substances to obtain supernatant, then performing membrane separation on the supernatant to obtain a solution containing active ingredients of Cistanche deserticola Ma, wherein the centrifugal speed is 4000 rpm/min; the membrane material selected for the membrane separation is one of polysulfone; and the pore size of the membrane separation is 0.8 microns.
(3) Performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) Drying the prepared controlled release formulation of the active ingredients of Cistanche deserticola Ma to obtain a dried controlled release product of the active ingredients of Cistanche deserticola Ma.

### EMBODIMENT 3

(1) Crushing and screening the raw material of Cistanche deserticola Ma to obtain powder of Cistanche deserticola Ma, mixing the powder of Cistanche deserticola Ma with appropriate amount of water, and then performing ultrasonic extraction to obtain extraction solution, wherein the extraction temperature of the ultrasonic extraction is 60° C., and the extraction time is 40 minutes.
(2) Performing centrifugation on the extraction solution to remove impurities and insoluble substances to obtain supernatant, then performing membrane separation on the supernatant to obtain a solution containing active ingredients of Cistanche deserticola Ma, wherein the centrifugal speed is 4000 rpm/min; the membrane material selected for the membrane separation is one of polysulfone; and the pore size of the membrane separation is 0.8 microns.
(3) Performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) Drying the prepared controlled release formulation of the active ingredients of Cistanche deserticola Ma to obtain a dried controlled release product of the active ingredients of Cistanche deserticola Ma.

### EMBODIMENT 4

(1) Crushing and screening the raw material of Cistanche deserticola Ma to obtain powder of Cistanche deserticola Ma, mixing the powder of Cistanche deserticola Ma with appropriate amount of water, and then performing ultrasonic extraction to obtain extraction solution, wherein the extraction temperature of the ultrasonic extraction is 50° C., and the extraction time is 50 minutes.
(2) Performing centrifugation on the extraction solution to remove impurities and insoluble substances to obtain supernatant, then performing membrane separation on the supernatant to obtain a solution containing active ingredients of Cistanche deserticola Ma, wherein the centrifugal speed is 4000 rpm/min; the membrane material selected for the membrane separation is one of polysulfone; and the pore size of the membrane separation is 0.8 microns.
(3) Performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) Drying the prepared controlled release formulation of the active ingredients of Cistanche deserticola Ma to obtain a dried controlled release product of the active ingredients of Cistanche deserticola Ma.

### EMBODIMENT 5

(1) Crushing and screening the raw material of Cistanche deserticola Ma to obtain powder of Cistanche deserticola Ma, mixing the powder of Cistanche deserticola Ma with appropriate amount of water, and then performing ultrasonic extraction to obtain extraction solution, wherein the extraction temperature of the ultrasonic extraction is 40° C., and the extraction time is 50 minutes.
(2) Performing centrifugation on the extraction solution to remove impurities and insoluble substances to obtain supernatant, then performing membrane separation on the supernatant to obtain a solution containing active ingredients of Cistanche deserticola Ma, wherein the centrifugal speed is 4000 rpm/min; the membrane material selected for the membrane separation is one of polysulfone; and the pore size of the membrane separation is 0.8 microns.
(3) Performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) Drying the prepared controlled release formulation of the active ingredients of Cistanche deserticola Ma to obtain a dried controlled release product of the active ingredients of Cistanche deserticola Ma.

### EMBODIMENT 6

(1) Crushing and screening the raw material of Cistanche deserticola Ma to obtain powder of Cistanche deserticola Ma, mixing the powder of Cistanche deserticola Ma with appropriate amount of water, and then performing ultrasonic extraction to obtain extraction solution, wherein the extraction temperature of the ultrasonic extraction is 45° C., and the extraction time is 60 minutes.
(2) Performing centrifugation on the extraction solution to remove impurities and insoluble substances to obtain supernatant, then performing membrane separation on the supernatant to obtain a solution containing active ingredients of Cistanche deserticola Ma, wherein the centrifugal speed is 4000 rpm/min; the membrane material selected for the membrane separation is one of polysulfone; and the pore size of the membrane separation is 0.8 microns.
(3) Performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma;
(4) Drying the prepared controlled release formulation of the active ingredients of Cistanche deserticola Ma to obtain a dried controlled release product of the active ingredients of Cistanche deserticola Ma.

Comparative example 1 and Comparative example 2 are extracted through existing technology.

By detecting the content of active ingredients in the aqueous extract of Cistanche deserticola Ma prepared in different embodiments and comparative examples, the content of echinoside, acteoside, iridoids, phenylethanoid glycosides, crude polysaccharides, total sugar, protein, amino acid, betaine, etc. in the aqueous extract are detected, respectively.

Wherein, the methods recorded in the pharmacopoeia were used for detection of echinoside and acteoside;

Phenylethanoid glycosides: It includes cistanoside A, tubuloside A, isoacteoside, 2′-acetylpiloside, and tubuloside B. The content of the above components was detected using the detection method in “Simultaneous Determination of 8 Components in Cistanches Herba Decoction Pieces by HPLC”;

Phenylethanol glycosides: It includes cistanoside A, tubuloside A, isoacteoside, 2′-acetylpiloside, and tubuloside B, echinoside, and acteoside; among them, the contents of cistanoside A, tubuloside A, isoacteoside, 2′-acetylpiloside, and tubuloside B were detected using the detection method in “Simultaneous Determination of 8 Components in Cistanches Herba Decoction Pieces by HPLC”.

Iridoids: it includes geniposidic acid and 8-epiloganic acid, the content of these components was detected using the detection method in “Simultaneous Determination of 8 Components in Cistanches Herba Decoction Pieces by HPLC”.

Crude polysaccharides: The first method disclosed in the first section of the “Detection Method for Functional Components of Health Food” for polysaccharide detection, namely “Phenol sulfuric acid spectrophotometric method for the determination of crude polysaccharides”, is used for measurement;
Amino acid: GB5009. 124-2016;
Total sugar: GB/T10782-2006;
Protein: GB5009. 5-2016 (first method);
Betaine: The content of betaine is determined by high-performance liquid chromatography. The chromatographic conditions of high-performance liquid chromatography are as follows: the mobile phase is acetonitrile-water with a ratio of 80:20, the detection wavelength is 196 nm, the column temperature is 30° C., and the flow rate is 1.0 mL/min.

| | | | | | | | Comparative | Comparative |
|---|---|---|---|---|---|---|---|---|
| | Embodiment 1 | Embodiment 2 | Embodiment 3 | Embodiment 4 | Embodiment 5 | Embodiment 6 | example 1 | example 2 |
| Echinacoside | 1010.2 | 1110.7 | 1013.4 | 1121.1 | 1010.3 | 1010.9 | 766.8 | 711.4 |
| mg/100 g | | | | | | | | |
| Acteoside | 42.1 | 43.2 | 49.4 | 39.2 | 39.9 | 41.6 | 10.7 | 9.6 |
| mg/100 g | | | | | | | | |
| Iridoid | 382.3 | 379.1 | 390.7 | 377.5 | 361.9 | 385.9 | 267.4 | 267.5 |
| mg/100 g | | | | | | | | |
| Phenylethanoid | 1455.3 | 1458.1 | 1399.6 | 1451.6 | 1455.8 | 1432.9 | 906.1 | 911.8 |
| glycosides | | | | | | | | |
| mg/100 g | | | | | | | | |
| Crude | 6.1 | 6.2 | 6.4 | 5.9 | 7.0 | 6.8 | 5.1 | 5.2 |
| polysaccharides | | | | | | | | |
| g/100 g | | | | | | | | |
| Total | 86.2 | 86.5 | 87.2 | 89.5 | 86.3 | 87.9 | 82.1 | 86.4 |
| Sugar | | | | | | | | |
| g/100 g | | | | | | | | |
| Protein | 11.8 | 11.6 | 11.7 | 12.1 | 10.9 | 12.1 | 10.2 | 10.9 |
| g/100 g | | | | | | | | |
| Amino acid | 0.7 | 0.7 | 0.7 | 0.7 | 0.7 | 0.7 | 0.7 | 0.7 |
| g/100 g | | | | | | | | |
| Betaine | 6.1 | 6.2 | 5.9 | 6.1 | 6.3 | 5.8 | 5.0 | 5.3 |
| g/100 g | | | | | | | | |

It can be seen that this disclosure can significantly improve the content of the main active ingredients in the controlled release formulation of the active ingredients of Cistanche deserticola Ma, especially the content of components such as acteoside, iridoid, total sugar, protein, amino acid, and betaine.

In summary, the present disclosure provides a production process for controlling the active ingredients of Cistanche deserticola Ma, which achieves efficient extraction and purification of the active ingredients of Cistanche deserticola Ma through steps such as ultrasonic extraction, centrifugal impurity removal, membrane separation, vacuum concentration, and drying, improving product quality and stability. By using ultrasonic extraction technology, it can improve the extraction efficiency and amount of active ingredients in Cistanche deserticola Ma under lower temperature and time conditions, reducing energy consumption and loss of active ingredients. By centrifugation and membrane separation steps, impurities and insoluble substances in the extraction solution can be effectively removed, improving the purity and quality stability of the product. By using vacuum concentration technology, a solution containing the active ingredients of Cistanche deserticola Ma can be quickly concentrated at lower temperatures, reducing the thermal decomposition and oxidation losses of the active ingredients, and improving the stability and shelf life of the product. By controlling the preparation of release formulations, slow release and long-lasting effects of the active ingredients in Cistanche deserticola Ma can be achieved, improving the efficacy and utilization of the product. By adopting drying technique, the controlled release product of active ingredients in Cistanche deserticola Ma can be quickly dried under lower temperature and vacuum conditions, ensuring the quality and stability of the product.

Finally, it should be noted that the above embodiments are only used to illustrate the technical solution of the present disclosure and not to limit it. Although the present disclosure has been described in detail with reference to the above embodiments, the ordinary skilled person in the art should understand that specific embodiments of the present disclosure can still be modified or equivalent substitution. Any modification or equivalent substitution without departing from the spirit and scope of the present disclosure should be covered within the scope of the claims of the present disclosure.

1. A production process for controlling the active ingredients of Cistanche deserticola Ma, comprising following steps:
(1) crushing and screening the raw material of Cistanche deserticola Ma to obtain powder of Cistanche deserticola Ma, mixing the powder of Cistanche deserticola Ma with a solvent, and then performing ultrasonic extraction to obtain extraction solution;
(2) performing centrifugation on the extraction solution to remove impurities and insoluble substances to obtain supernatant, then performing membrane separation on the supernatant to obtain a solution containing active ingredients of Cistanche deserticola Ma,
(3) performing vacuum concentration on the solution containing the active ingredients of Cistanche deserticola Ma to obtain a concentrated solution, then mixing the concentrated solution with excipients and stirring evenly to prepare a controlled release formulation for the active ingredients of Cistanche deserticola Ma,
(4) drying the prepared controlled release formulation of the active ingredients of Cistanche deserticola Ma to obtain a dried controlled release product of the active ingredients of Cistanche deserticola Ma.

2. The production process for controlling the active ingredients of Cistanche deserticola Ma according to claim 1, wherein extraction temperature of the ultrasonic extraction is 40-60° C., and extraction time is 30-60 minutes.

3. The production process for controlling the active ingredients of Cistanche deserticola Ma according to claim 1, wherein membrane material selected for the membrane separation is one of polysulfone, polyethersulfone, or polyamide.

4. The production process for controlling the active ingredients of Cistanche deserticola Ma according to claim 1, wherein a pore size of the membrane separation is selected between 0.1 and 10 microns.

5. The production process for controlling the active ingredients of Cistanche deserticola Ma according to claim 1, wherein centrifugal speed is 4000 rpm/min.

6. The production process for controlling the active ingredients of Cistanche deserticola Ma according to claim 1, wherein concentration temperature of the vacuum concentration is 65° C., and a vacuum degree during concentration is above −0.08 Mpa.

7. The production process for controlling the active ingredients of Cistanche deserticola Ma according to claim 1, wherein drying temperature is 70° C., and a vacuum degree during drying is above −0.08 Mp.

8. The production process for controlling the active ingredients of Cistanche deserticola Ma according to claim 1, wherein the solvent is one selected from the group consisting of water, ethanol, methanol, ethyl acetate, n-butanol, chloroform, and dimethyl sulfoxide.
